(19) United States
(12) Patent Application Publication
(10) Pub. No.: US 2022/0015844 A1
(43) Pub. Date: Jan. 20, 2022

(54) KINEMATICS OF WRISTED LAPAROSCOPIC INSTRUMENTS

(71) Applicant: Activ Surgical, Inc., Boston, MA (US)

(72) Inventor: Hossein Dehghani, Boston, MA (US)

(21) Appl. No.: 17/356,181

(22) Filed: Jun. 23, 2021

Related U.S. Application Data
(63) Continuation of application No. PCT/US2019/068765, filed on Dec. 27, 2019.
(60) Provisional application No. 62/785,979, filed on Dec. 28, 2018.

(51) Int. Cl. A61B 34/32; A61B 34/10

(52) U.S. Cl. CPC A61B 34/32; A61B 34/10; A61B 2034/302; A61B 2034/107; A61B 2034/105

(57) ABSTRACT

Systems, methods, and computer program products for determining joint angles of a wristed laparoscopic tool of a robotic surgical instrument are disclosed. The method includes providing a first robotic arm where the robotic arm includes surgical instrument having a proximal end and a distal end and the tool is disposed at the distal end of the instrument. The tool has an elbow joint, a wrist joint, and a distal-most end. A three-dimensional position of the wrist joint is determined based on a three-dimensional position and orientation of the distal-most end. A three dimensional position of the elbow joint is determined based on the three-dimensional position of the wrist joint. A first angle corresponding to a twist of the tool, a second angle corresponding to an elbow joint angle of the tool, a third angle corresponding to a wrist angle, and a fourth angle corresponding to an open/close angle of the tool are determined.

### CROSS-REFERENCE TO RELATED APPLICATION

This application is a continuation application of International Application No. PCT/US2019/068765, filed on Dec. 27, 2019, which application claims the benefit of U.S. Provisional Patent Application No. 62/785,979, filed on Dec. 28, 2018, which applications are incorporated herein by reference in their entirety for all purposes.

### BACKGROUND

Embodiments of the present disclosure generally relate to kinematics of wristed laparoscopic tools of robotic surgical instruments. In particular, the present disclosure describes the determination of joint angles and/or pose in a wristed laparoscopic tool based on forward and/or reverse kinematics.

### BRIEF SUMMARY

According to embodiments of the present disclosure, systems for, methods for, and computer program products for determining joint angles of a wristed laparoscopic tool of a surgical instrument are provided. In various embodiments, a system includes a first robotic arm having a proximal end and a distal end. The proximal end is fixed to a base. The system further includes a surgical instrument having a proximal end and a distal end where the surgical instrument is disposed at the distal end of the robotic arm. The surgical instrument is configured to be inserted into a trocar positioned in an incision in a patient. The system further includes a wristed laparoscopic tool coupled to the distal end of the surgical instrument. The wristed laparoscopic tool has an elbow joint, a wrist joint, and a distal-most end. The system further includes a computing node comprising computer readable storage medium having program instructions embodied therewith. The program instructions are executable by a processor of the computing node to cause the processor to perform a method where a three-dimensional position of the wrist joint is determined based on a three-dimensional position and orientation of the distal-most end, a three-dimensional position of the elbow joint based on the three-dimensional position of the wrist joint, and a first angle corresponding to a twist of the tool, a second angle corresponding to an elbow joint angle of the tool, a third angle corresponding to a wrist angle, and a fourth angle corresponding to an open/close angle of the tool are determined. The first angle, the second angle, the third angle, and the fourth angle are determined based on a three-dimensional position of the trocar, the three-dimensional position and orientation of the distal-most end, the three-dimensional position of the wrist joint, and the three-dimensional position of the elbow joint.

In various embodiments, the three-dimensional position of the wrist joint is determined by P_{Wrist}=P_{Tip}−L1(\vec{u}) where P_{wrist} corresponds to the three-dimensional position of the wrist joint, P_{Tip} corresponds to the three-dimensional position of the distal end, L1 corresponds to the linear distance between the distal end and the wrist joint, and \vec{u} corresponds to a directional unit vector pointing from the distal-most end towards the wrist joint. In various embodiments, the three-dimensional position of the elbow joint is determined based on an intersection of a circle centered at the three-dimensional position of the wrist joint and a plane defined by a first vector representing a rotation axis of the wrist joint and a second vector representing a line connecting the wrist joint to the incision. In various embodiments, the circle includes a radius equal to a linear distance between the three-dimensional position of the wrist joint and the three-dimensional position of the elbow joint. In various embodiments, the intersection of the circle and the plane defines two potential solutions. In various embodiments, the method further includes selecting one of the two potential solutions that does not violate joint limits of the wristed laparoscopic tool. In various embodiments, the method further includes determining a vector normal to the plane.

In various embodiments, the first angle is determined based on: θ₁=a tan(^{Tip}T_{Elbow} (2,1), ^{TiP}T_{Elbow}(1,1)), where θ₁ is the first angle, ^{Tip} T_{Elbow} is the transformation matrix of the elbow joint with respect to the distal-most end. In various embodiments, the second angle is determined based on:

$${θ}_{2} = acos ( \frac{{P}_{Wrist} - {P}_{Elbow}}{\vert {P}_{Wrist} - {P}_{Elbow} \vert} , \frac{{P}_{Trocar} - {P}_{Elbow}}{\vert {P}_{Trocar} - {P}_{Elbow} \vert} ) ,$$

where θ₂ is the second angle, P_{Wrist} is the three-dimensional position of the wrist joint, P_{Elbow} is the three-dimensional position of the elbow joint, and P_{Trocar} is a three-dimensional position of the trocar. In various embodiments, the third angle is determined based on:

$$third angle = \frac{{θ}_{4} + {θ}_{3}}{2} = acos ( \frac{{P}_{Elbow} - {P}_{Wrist}}{\vert {P}_{Elbow} - {P}_{Wrist} \vert} , \frac{{P}_{Tip} - {P}_{Wrist}}{\vert {P}_{Tip} - {P}_{Wrist} \vert} ) ,$$

where θ₃ is an angle from a line defined from the three-dimensional position of the elbow to the three-dimensional position of the wrist and a first gripping member of the tool, θ₄ is an angle from the line defined from the three-dimensional position of the elbow to the three-dimensional position of the wrist and a second gripping member of the tool, P_{Wrist} is the three-dimensional position of the wrist joint, and P_{Elbow} is the three-dimensional position of the elbow joint. In various embodiments, the fourth angle is determined based on:

$$fourth angle = \frac{{θ}_{3} - {θ}_{4}}{2}$$

where θ₃ is an angle from a line defined from the three-dimensional position of the elbow to the three-dimensional position of the wrist and a first gripping member of the tool, θ₄ is an angle from the line defined from the three-dimensional position of the elbow to the three-dimensional position of the wrist and a second gripping member of the tool.

In various embodiments, a method for determining joint angles of a wristed laparoscopic tool of a surgical instrument is provided. The method includes providing a first robotic arm where the robotic arm includes a surgical instrument having a proximal end and a distal end. The tool is disposed at the distal end of the surgical instrument and has an elbow joint, a wrist joint, and a distal-most end. The surgical instrument is configured to be inserted into a trocar positioned in an incision in a patient. A three-dimensional position of the wrist joint is determined based on a three-dimensional position and orientation of the distal-most end. A three dimensional position of the elbow joint is determined based on the three-dimensional position of the wrist joint. A first angle corresponding to a twist of the tool, a second angle corresponding to an elbow joint angle of the tool, a third angle corresponding to a wrist angle, and a fourth angle corresponding to an open/close angle of the tool are determined. The first angle, the second angle, the third angle, and the fourth angle are determined based on a three-dimensional position of the trocar, the three-dimensional position and orientation of the distal-most end, the three-dimensional position of the wrist joint, and the three dimensional position of the elbow joint.

In various embodiments, the three-dimensional position of the wrist joint is determined by P_{Wrist}=P_{Tip}−L1(\vec{u}) where P_{Wrist} corresponds to the three-dimensional position of the wrist joint, P_{Ttp} corresponds to the three-dimensional position of the distal end, L1 corresponds to the linear distance between the distal end and the wrist joint, and \vec{u} corresponds to a directional unit vector pointing from the distal-most end towards the wrist joint. In various embodiments, the three-dimensional position of the elbow joint is determined based on an intersection of a circle centered at the three-dimensional position of the wrist joint and a plane defined by a first vector representing a rotation axis of the wrist joint and a second vector representing a line connecting the wrist joint to the incision. In various embodiments, the circle includes a radius equal to a linear distance between the three-dimensional position of the wrist joint and the three-dimensional position of the elbow joint. In various embodiments, the intersection of the circle and the plane defines two potential solutions. In various embodiments, the method further includes selecting one of the two potential solutions that does not violate joint limits of the wristed laparoscopic tool. In various embodiments, the method further includes determining a vector normal to the plane.

In various embodiments, the first angle is determined based on: θ₁=a tan(^{Tip}T_{Elbow} (2,1), ^{TiP}T_{Elbow}(1,1)), where θ₁ is the first angle, ^{Tip}T_{Elbow} is the transformation matrix of the elbow joint with respect to the distal-most end. In various embodiments, the second angle is determined based on:

$${θ}_{2} = acos ( \frac{{P}_{Wrist} - {P}_{Elbow}}{\vert {P}_{Wrist} - {P}_{Elbow} \vert} , \frac{{P}_{Trocar} - {P}_{Elbow}}{\vert {P}_{Trocar} - {P}_{Elbow} \vert} ) ,$$

where θ₂ is the second angle, P_{Wrist} is the three-dimensional position of the wrist joint, P_{Elbow} is the three-dimensional position of the elbow joint, and P_{Trocar} is a three-dimensional position of the trocar. In various embodiments, the third angle is determined based on:

$$third angle = \frac{{θ}_{4} + {θ}_{3}}{2} = acos ( \frac{{P}_{Elbow} - {P}_{Wrist}}{\vert {P}_{Elbow} - {P}_{Wrist} \vert} , \frac{{P}_{Tip} - {P}_{Wrist}}{\vert {P}_{Tip} - {P}_{Wrist} \vert} ) ,$$

where θ₃ is an angle from a line defined from the three-dimensional position of the elbow to the three-dimensional position of the wrist and a first gripping member of the tool, θ₄ is an angle from the line defined from the three-dimensional position of the elbow to the three-dimensional position of the wrist and a second gripping member of the tool, P_{Wrist} is the three-dimensional position of the wrist joint, and P_{Elbow} is the three-dimensional position of the elbow joint. In various embodiments, the fourth angle is determined based on:

$$fourth angle = \frac{{θ}_{3} - {θ}_{4}}{2}$$

where θ₃ is an angle from a line defined from the three-dimensional position of the elbow to the three-dimensional position of the wrist and a first gripping member of the tool, θ₄ is an angle from the line defined from the three-dimensional position of the elbow to the three-dimensional position of the wrist and a second gripping member of the tool.

In various embodiments, a computer program product for determining joint angles of a wristed laparoscopic tool of a surgical instrument is provided in the form of a computer readable storage medium having program instructions embodied therewith. The program instructions are executable by a processor to cause the processor to perform a method where a three-dimensional position of a wrist joint of a wristed laparoscopic tool of a surgical instrument is determined based on a three-dimensional position and orientation of a distal-most end of the tool. The surgical instrument is configured to be inserted into a trocar positioned in an incision in a patient. A three dimensional position of an elbow joint of the tool is determined based on the three-dimensional position of the wrist joint. A first angle corresponding to a twist of the tool, a second angle corresponding to an elbow joint angle of the tool, a third angle corresponding to a wrist angle, and a fourth angle corresponding to an open/close angle of the tool are determined. The first angle, the second angle, the third angle, and the fourth angle are determined based on a three-dimensional position of the trocar, the three-dimensional position and orientation of the distal-most end, the three-dimensional position of the wrist joint, and the three dimensional position of the elbow joint.

In various embodiments, the three-dimensional position of the wrist joint is determined by P_{Wrist}=P_{Tip}−L1(\vec{u}) where P_{Wrist} corresponds to the three-dimensional position of the wrist joint, P_{Tip} corresponds to the three-dimensional position of the distal end, L1 corresponds to the linear distance between the distal end and the wrist joint, and \vec{u} corresponds to a directional unit vector pointing from the distal-most end towards the wrist joint. In various embodiments, the three-dimensional position of the elbow joint is determined based on an intersection of a circle centered at the three-dimensional position of the wrist joint and a plane defined by a first vector representing a rotation axis of the wrist joint and a second vector representing a line connecting the wrist joint to the incision. In various embodiments, the circle includes a radius equal to a linear distance between the three-dimensional position of the wrist joint and the three-dimensional position of the elbow joint. In various embodiments, the intersection of the circle and the plane defines two potential solutions. In various embodiments, the method further includes selecting one of the two potential solutions that does not violate joint limits of the wristed laparoscopic tool. In various embodiments, the method further includes determining a vector normal to the plane.

In various embodiments, the first angle is determined based on: θ₁=a tan(^{Tip}T_{Elbow} (2,1), ^{TiP}T_{Elbow}(1,1)), where θ₁ is the first angle, ^{Tip}T_{Elbow} is the transformation matrix of the elbow joint with respect to the distal-most end. In various embodiments, the second angle is determined based on:

$${θ}_{2} = acos ( \frac{{P}_{Wrist} - {P}_{Elbow}}{\vert {P}_{Wrist} - {P}_{Elbow} \vert} , \frac{{P}_{Trocar} - {P}_{Elbow}}{\vert {P}_{Trocar} - {P}_{Elbow} \vert} ) ,$$

where θ₂ is the second angle, P_{Wrist} is the three-dimensional position of the wrist joint, P_{Elbow} is the three-dimensional position of the elbow joint, and P_{Trocar} is a three-dimensional position of the trocar. In various embodiments, the third angle is determined based on:

$$third angle = \frac{{θ}_{4} + {θ}_{3}}{2} = acos ( \frac{{P}_{Elbow} - {P}_{Wrist}}{\vert {P}_{Elbow} - {P}_{Wrist} \vert} , \frac{{P}_{Tip} - {P}_{Wrist}}{\vert {P}_{Tip} - {P}_{Wrist} \vert} ) ,$$

where θ₃ is an angle from a line defined from the three-dimensional position of the elbow to the three-dimensional position of the wrist and a first gripping member of the tool, θ₄ is an angle from the line defined from the three-dimensional position of the elbow to the three-dimensional position of the wrist and a second gripping member of the tool, P_{Wrist} is the three-dimensional position of the wrist joint, and P_{Elbow} is the three-dimensional position of the elbow joint. In various embodiments, the fourth angle is determined based on:

$$fourth angle = \frac{{θ}_{3} - {θ}_{4}}{2}$$

where θ₃ is an angle from a line defined from the three-dimensional position of the elbow to the three-dimensional position of the wrist and a first gripping member of the tool, θ₄ is an angle from the line defined from the three-dimensional position of the elbow to the three-dimensional position of the wrist and a second gripping member of the tool.

### BRIEF DESCRIPTION OF THE DRAWINGS

FIG. 1 illustrates a surgical instrument having a wristed laparoscopic tool for performing robotic laparoscopic surgery according to an embodiment of the present disclosure.

FIG. 2A illustrates a first step in determining joint angles for a wristed laparoscopic tool according to an embodiment of the present disclosure.

FIGS. 2B-2C illustrates a second step in determining joint angles for a wristed laparoscopic tool according to an embodiment of the present disclosure.

FIG. 2D illustrates various joint angles on a wristed laparoscopic tool according to an embodiment of the present disclosure.

FIG. 3 illustrates a robotic arm system for performing laparoscopic surgery according to an embodiment of the present disclosure.

FIG. 4 illustrates a flowchart of a method for determining joint angles of a wristed laparoscopic tool according to an embodiment of the present disclosure.

FIG. 5 depicts an exemplary computing node according to various embodiments of the present disclosure.

### DETAILED DESCRIPTION

Many surgical maneuvers (e.g., suturing, cutting, and/or folding) require highly dexterous and highly accurate motion of surgical tools to achieve a satisfactory surgical outcome. In fully automated robotic surgical procedures, surgical robots generally include a surgical instrument attached thereto having a tool that is inserted through a trocar placed in a small, keyhole incision in the abdomen of a patient. A keyhole incision, as used herein, may refer to a minimally invasive incision that is about 0.25 inch to 1 inch in size. The tool may include any suitable medical tool, such as, for example, a camera, a cutting tool, a gripping tool, a crimping tool, an electrocautery tool, or any other suitable tool as is known in the art. When the surgical instrument is inserted through the trocar (and into a body cavity, e.g., abdomen, of the patient), the orientation (including the angles between each of the joints of the tool) of the wristed laparoscopic tool is not visible to a surgeon. Knowing the joint angles of the wristed laparoscopic tool is critically important to a surgical procedure as the tool performs complex maneuvers in a small space within a surgical site. In various embodiments, the joint angles may be determined from known values, such as, for example, the three-dimensional position of the trocar and the pose of the tool (i.e., the three dimensional position and orientation of the distal-most end of the tool). In various embodiments, the pose of the tool may be determined from known values, such as, for example, the three-dimensional position of the trocar and the joint angles.

Accordingly, a need exists for a system and method to determine joint angles of a wristed laparoscopic tool to thereby enable accurate surgical maneuvers and improve robotic-assisted surgery.

In various embodiments, the three-dimensional position of the trocar (through which a wristed laparoscopic tool is inserted) is generally known and may be denoted as P_{trocar}=[x_{trocar} y_{trocar} z_{trocar}]^{T} in three-dimensional Cartesian coordinates. In various embodiments, a distal end (for example the distal-most tip of the wristed laparoscopic tool) has 6 degrees of freedom and is denoted as: (xₜᵢₚ, yₜᵢₚ, zₜᵢₚ, αₜᵢₚ, βₜᵢₚ, γₜᵢₚ), where αₜᵢₚ, βₜᵢₚ, γₜᵢₚ are Euler angles (i.e., roll, yaw, and pitch) representing the orientation of the distal end. The three-dimensional position of the tip may be represented as:

Pₜᵢₚ=[xₜᵢₚyₜᵢₚzₜᵢₚ]^{T}

The three-dimensional orientation of the tip may be represented as:

$${R}_{tip} = [ \vec{u} \vec{v} \vec{w} ] = [ \begin{matrix}{u}_{1} & {v}_{1} & {w}_{1} \\ {u}_{2} & {v}_{2} & {w}_{2} \\ {u}_{3} & {v}_{3} & {w}_{3}\end{matrix} ] = {R}_{z} ( α ) {R}_{y} ( β ) {R}_{x} ( γ )$$

where \vec{u}, is the unit vector along the x-axis, \vec{v} is the unit vector along the y-axis, and \vec{w} is the unit vector along the z-axis, R_{z} is the 3D rotation matrix about the z-axis by alpha, R_{y} is the 3D rotation matrix about the y-axis by beta, and Rx is the 3D rotation matrix about the x-axis by gamma.

In various embodiments, a pose of the tool includes the position of the tip and the rotation (which may be expressed by Euler angles or a 3×3 rotation matrix). In various embodiments, when the rotation is expressed in a matrix form, the first column, the second column, and the third column may represent the unit vector along the x-axis, y-axis, and z-axis, respectively. In various embodiments, the pose can be expressed as (x, y, z, roll, pitch, yaw) or a 4×4 transformation matrix. The distal end position and orientation (i.e., the pose) may be represented as the following matrix:

$${}_{}^{b}{T}{}_{tip}^{} = [ \begin{matrix}{[ R ]}_{3 \times 3} & [ \begin{matrix}{x}_{tip} \\ {y}_{tip} \\ {z}_{tip}\end{matrix} ] \\ 0 & 1\end{matrix} ] = [ \begin{matrix}{u}_{1} & {v}_{1} & {w}_{1} & {x}_{tip} \\ {u}_{2} & {v}_{2} & {w}_{2} & {y}_{tip} \\ {u}_{3} & {v}_{3} & {w}_{3} & {z}_{tip} \\ 0 & 0 & 0 & 1\end{matrix} ]$$

where Tₜᵢₚ is the transformation matrix representing the pose of the tip (i.e., position and orientation).

The Euler angles of the tip (i.e., αₜᵢₚ, βₐₚ, γₜᵢₚ) may be represented in a 3×3 matrix represented as [R]_{3×3}.

Joint angles of the wristed laparoscopic tool (e.g., a grasper) may be computed knowing the three-dimensional positional information of the trocar and the three-dimensional position and orientation of the distal end of the wristed laparoscopic tool (e.g., grasper). For example, the twist (θ₁) of the grasper, the elbow angle (θ₂), the wrist angle

$$( \frac{{θ}_{3} - {θ}_{4}}{2} )$$

and the open/close angle

$$( \frac{{θ}_{3} + {θ}_{4}}{2} ) ,$$

may be determined. In various embodiments, the twist angle is a first joint angle. In various embodiments, the elbow joint angle is a second joint angle. In various embodiments, the wrist angle is a third joint angle and is determined using θ₃ and θ₄. In various embodiments, the open/close angle is a fourth joint angle and is determined using θ₃ and θ₄. In various embodiments, the wrist angle includes the angle between a first axis defined from the three-dimensional position of the elbow joint to the three-dimensional position of the wrist joint and a second axis including the three-dimensional position of the wrist joint and extending halfway between the grasper jaws. In various embodiments, the open/close angle is the angle between a single grasper jaw and an axis including the three-dimensional position of the wrist joint and extending halfway between the grasper jaws. In various embodiments, twice the open/close angle defines the angle between the two grasper jaws.

Additionally, the pose of the proximal end of the instrument shaft may be determined. In various embodiments, with six degrees of freedom, the pose may be represented as (xₚᵣₒₓᵢₘₐₗ, yₚᵣₒₓᵢₘₐₗ, zₚᵣₒₓᵢₘₐₗ, αₚᵣₒₓᵢₘₐₗ, βₚᵣₒₓᵢₘₐₗ, γₚᵣₒₓᵢₘₐₗ). In various embodiments, any point along a rigid shaft of the surgical instrument may be considered the proximal end of the instrument, and the pose of that point may be determined. In various embodiments, the proximal end may be the point where the surgical instrument is attached to the distal end of the robotic arm.

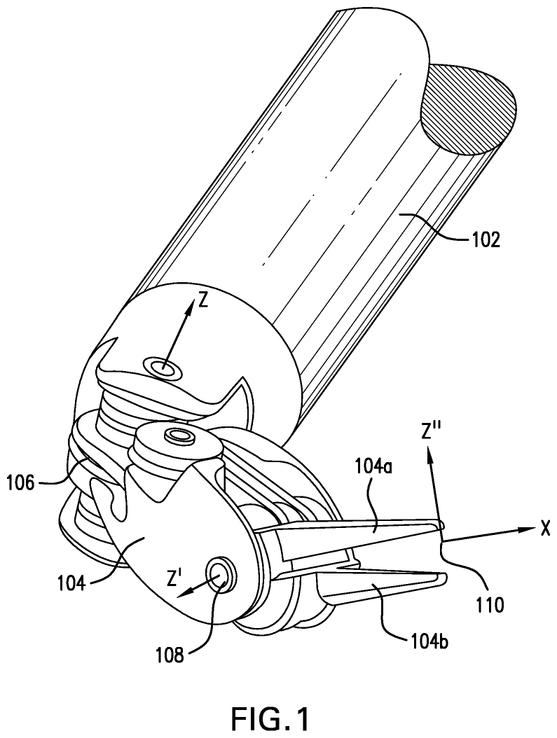

FIG. 1 illustrates a surgical instrument 102 having a wristed laparoscopic tool 104 for performing robotic laparoscopic surgery according to an embodiment of the present disclosure. The wristed laparoscopic tool 104 includes a grasper having two gripping members 104a, 104b. The wristed laparoscopic tool 104 is coupled to the surgical instrument 102 at a rotatable elbow joint 106 and the gripping members 104a, 104b are rotatably coupled to the wristed laparoscopic tool 104 at a rotatable wrist joint 108. As shown in FIG. 1, elbow joint 106 is rotatable about a Z-axis and wrist joint 108 is rotatable about a Z′-axis. The distal end 110 of the gripping members 104a, 104b includes an X-axis (extending longitudinally along the closed gripping members) and a Z″-axis (normal to the X-axis).

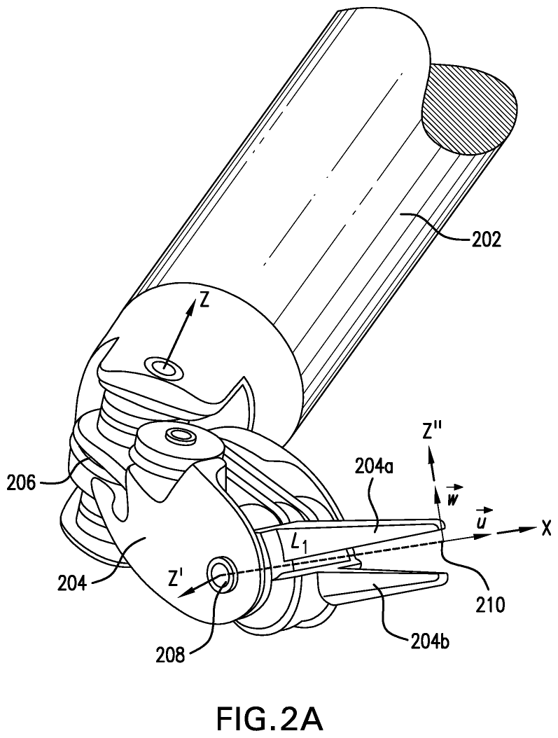

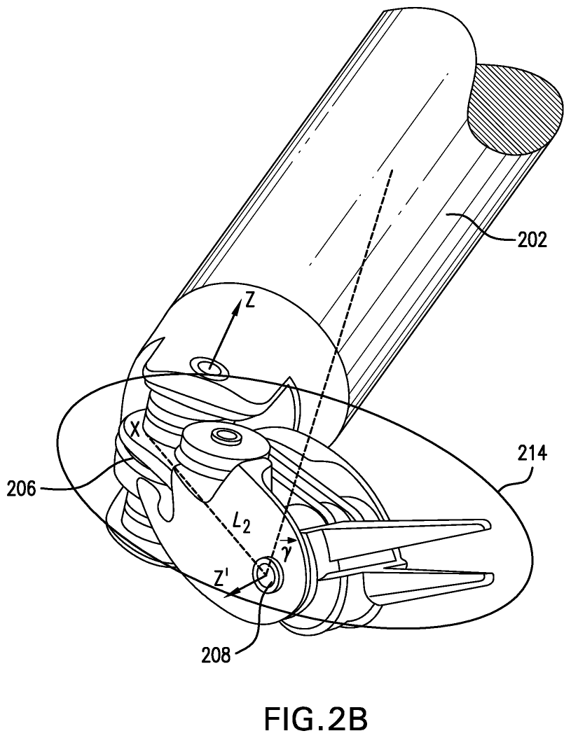

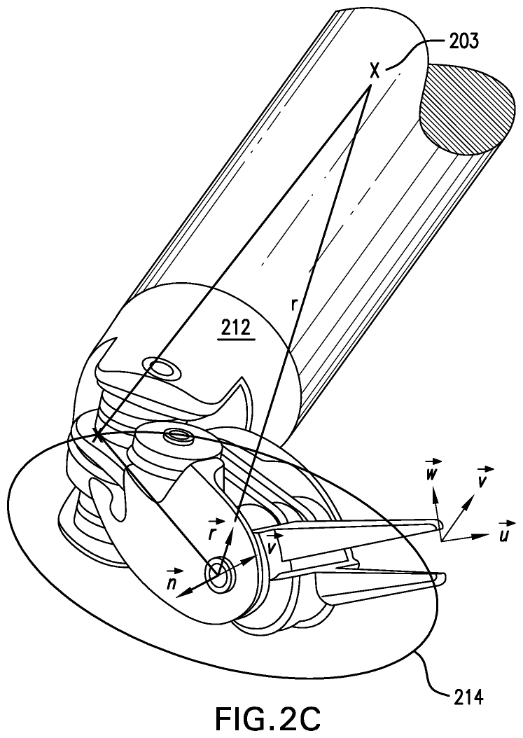

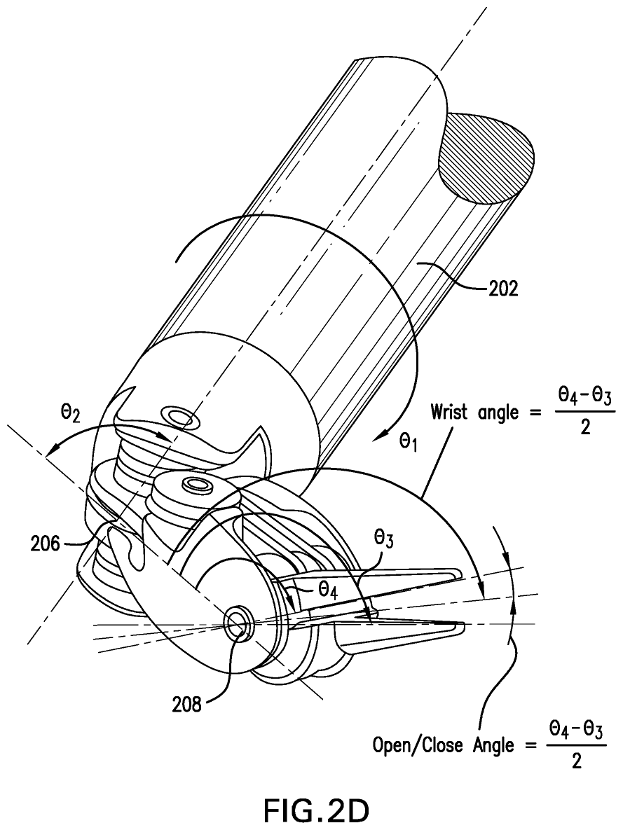

FIG. 2A illustrates a first step in determining joint angles for a tool 204 according to an embodiment of the present disclosure. Similar to FIG. 1, a surgical instrument 202 includes a wristed laparoscopic tool 204 at the distal end of the surgical instrument 202 via an elbow joint 206. The surgical instrument 204 is coupled to the gripping members 204a, 204b via a wrist joint 208. The gripping members 204a, 204b have a distal end 210 that is the distal-most point of the gripping members 204a, 204b. In the first step, the three-dimensional position of the wrist joint, represented as P_{Wrist}=[x_{Wrist} y_{Wrist} z_{Wrist}]^{T}, is computed as follows:

P_{Wrist}=P_{Tip}−L1(\vec{u})

where P_{Wrist} corresponds to the three-dimensional position of the wrist joint 208,
P_{Tip} corresponds to the three-dimensional position of the distal end 210, L1 corresponds to the linear distance between the distal end 210 and the wrist joint 208, and \vec{u} corresponds to a directional unit vector pointing from the distal end 210 towards the wrist joint 208. Because the position of the distal end 210 is known, the wrist joint 208 is offset by the length of the gripping members 204a, 204b along an axis (i.e., the X-axis). In various embodiments, \vec{u} may represent the first column of [R]_{3×3}.

FIGS. 2B-2C illustrates a second step in determining joint angles for a wristed laparoscopic tool 204 according to an embodiment of the present disclosure. In the second step, the three-dimensional position of the elbow joint 206 is computed, represented as P_{Elbow}=[x_{Elbow} y_{Elbow} z_{Elbow}]^{T}. The three dimensional position of the elbow may be computed from the intersection of a 2D plane 212 with a circle 214.

In various embodiments, the circle 214 is centered at the three-dimensional position of the wrist joint 208 and has a radius of L2. In various embodiments, L2 is a geometric parameter that represents the linear distance between the wrist joint 208 and the elbow joint 206. In various embodiments, the circle 214 has a vector \vec{v} that is normal to the circle 214.

In various embodiments, the 2D plane 212 is a 2D surface containing the three-dimensional positions of the trocar 212, elbow joint 206, and wrist joint 208. In various embodiments, two vectors of this plane are known \vec{v} and \vec{r}. In various embodiments, the vector \vec{v} represents the rotation axis of the wrist (\vec{v} is parallel to the y-axis of the tip and, thus, can be obtained from the tip rotation matrix) and the vector \vec{r} represents the line connecting the wrist joint 208 to the trocar. In various embodiments, the plane 212 includes a normal vector \vec{n} that includes the three-dimensional position of the wrist joint. In various embodiments, the normal vector to the 2D plane can be calculated as the cross-product of u and v as follows:

$$\vec{r} = \frac{{P}_{Trocar} - {P}_{Wrist}}{‖ {P}_{Trocar} - {P}_{Wrist} ‖}$$

$$\vec{n} = \vec{r} \times \vec{v} = \vert \begin{matrix}i & j & k \\ {r}_{1} & {r}_{2} & {r}_{3} \\ {v}_{1} & {v}_{2} & {v}_{3}\end{matrix} \vert$$

In various embodiments, the point-normal form of the plane may be defined using \vec{r} and \vec{v} such that a plane with normal vector \vec{n}=\vec{v}×\vec{r} can be constructed. In various embodiments, \vec{n} is a unit vector of the plane 212 in which the three-dimensional position of the trocar, the three-dimensional position of the elbow joint, and the three-dimensional position of the wrist joint belong. In various embodiments, \vec{n} (and the plane 212) is independent of joint angles.

In various embodiments, the three-dimensional position of the elbow includes an intersection of both the circle and the plane. In various embodiments, the intersection between the circle 214 and the 2D plane 212 results in two solutions. However, one of the solutions violates the joint limits and, thus, can be eliminated, leaving the correct solution.

In a final step, the joint angles, as described above, may be computed from the three-dimensional positional information of the surgical instrument 202, elbow joint 206, and wrist joint 208, and the three-dimensional position and orientation information of the distal end 210. In various embodiments, the joint angles are computed using geometric relationships in three-dimensional space.

FIG. 2D illustrates various joint angles on a wristed laparoscopic tool according to an embodiment of the present disclosure. In various embodiments, the various joint angles may be determined as follows:

$${θ}_{2} = acos ( \frac{{P}_{Wrist} - {P}_{Elbow}}{\vert {P}_{Wrist} - {P}_{Elbow} \vert} , \frac{{P}_{Trocar} - {P}_{Elbow}}{\vert {P}_{Trocar} - {P}_{Elbow} \vert} )$$

$$\frac{{θ}_{3} + {θ}_{4}}{2} = acos ( \frac{{P}_{Elbow} - {P}_{Wrist}}{\vert {P}_{Elbow} - {P}_{Wrist} \vert} , \frac{{P}_{Tip} - {P}_{Wrist}}{\vert {P}_{Tip} - {P}_{Wrist} \vert} )$$

$${}_{}^{b}{T}{}_{Elbow}^{} = {}_{}^{b}{T}{}_{tip}^{} {}_{}^{Tip}{T}{}_{Elbow}^{}$$

$${}_{}^{Tip}{T}{}_{Elbow}^{} = {}_{}^{Tip}{T}{}_{Elbow}^{- 1}$$

$${θ}_{1} = atan ( {}_{}^{Tip}{T}{}_{Elbow}^{} ( 2 , 1 ) , {}_{}^{Tip}{T}{}_{Elbow}^{} ( 1 , 1 ) )$$

In various embodiments, when the joint angles (θ₁, θ₂, θ₃, θ₄) and the three-dimensional position of the trocar are known, the pose of the tool may be determined. In various embodiments, this process may be called forward kinematics. In various embodiments, the trocar position P_{trocar}=[x_{trocar} y_{trocar} z_{trocar}]^{T} may be determined. For example, the trocar position may be specified by a surgeon. In various embodiments, the trocar position may be provided by a trajectory planning algorithm.

In various embodiments, the elbow angle (θ₂), the wrist angle (θ₃), the open/close angle (θ₄), and the position of the trocar are known and, thus, the position of the elbow joint and the position of the wrist joint may be determined based on the following relationship:

$${θ}_{2} = acos ( \frac{{P}_{Wrist} - {P}_{Elbow}}{\vert {P}_{Wrist} - {P}_{Elbow} \vert} , \frac{{P}_{Trocar} - {P}_{Elbow}}{\vert {P}_{Trocar} - {P}_{Elbow} \vert} )$$

$$\frac{{θ}_{3} + {θ}_{4}}{2} = acos ( \frac{{P}_{Elbow} - {P}_{Wrist}}{\vert {P}_{Elbow} - {P}_{Wrist} \vert} , \frac{{P}_{Tip} - {P}_{Wrist}}{\vert {P}_{Tip} - {P}_{Wrist} \vert} )$$

In various embodiments, the transformation matrix from the elbow to the tip may be computed based on the following relationship:

^{b}T_{Elbow}=^{b}Tₜᵢₚ^{Tip}T_{Elbow}

^{Tip}Elbow=^{Tip}T_{Elbow}⁻¹

θ₁=a tan(^{TiP}T_{Elbow}(2,1),^{Tip}T_{Elbow}(1,1))

where ^{TiP}T_{Elbow} is the transformation matrix from elbow to the tip and is computable when the elbow and wrist angles are known. In various embodiments, the pose of the tool ^{b}Tₜᵢₚ may also be determined from the above relationship.

In various embodiments, the pose of the tool may be expressed in Cartesian coordinates having six (6) degrees of freedom (xₜᵢₚ, yₜᵢₚ, zₜᵢₚ, αₜᵢₚ, βₜᵢₚ, γₜᵢₚ). In various embodiments, the pose of the tool may be expressed as a 4×4 transformation matrix as follows:

$${}_{}^{b}{T}{}_{tip}^{} = [ \begin{matrix}{u}_{1} & {v}_{1} & {w}_{1} & {x}_{tip} \\ {u}_{2} & {v}_{2} & {w}_{2} & {y}_{tip} \\ {u}_{3} & {v}_{3} & {w}_{3} & {z}_{tip} \\ 0 & 0 & 0 & 1\end{matrix} ]$$

In various embodiments, the 3D position of the wrist P_{wrist}=[x_{Wrist} y_{Wrist} z_{Wrist}]^{T} can be computed based on the tip pose and the trocar position as follows:

P_{Wrist}=P_{Tip}−L₁(\vec{u})

In various embodiments, the three-dimensional position of elbow is determined from P_{Elbow}=[x_{Elbow} y_{Elbow} z_{Elbow}]^{T}. In various embodiments, the three-dimensional position of the elbow is the intersection of a circle and a plane in three-dimensional space, as described above. In various embodiments, the circle is centered at P_{Wrist} with radius L₂ and located on a plane with a normal vector \vec{v}. In various embodiments, the plane is constructed in the point-normal form. In various embodiments, the plane passes through the wrist (P_{Wrist}) and has a unit vector n. In various embodiments, the unit vector \vec{n} is the cross product of the vector \vec{v} and \vec{r}. In various embodiments, the unit vector \vec{r} is an auxiliary vector connecting P_{Wrist} to P_{Trocar}.

In various embodiments, a control system may receive the position information of the joints, the angles, and/or the pose. In various embodiments, the control system may receive commands to move one or more of the joints to a target angle to thereby actuate the surgical instrument. In various embodiments, the control system may receive commands to move three-dimensional positions (wrist joint, elbow joint, and/or distal tip of tool) to a target position to thereby actuate the surgical instrument. In various embodiments, the control system may receive commands to move the tool to a target pose to thereby actuate the surgical instrument.

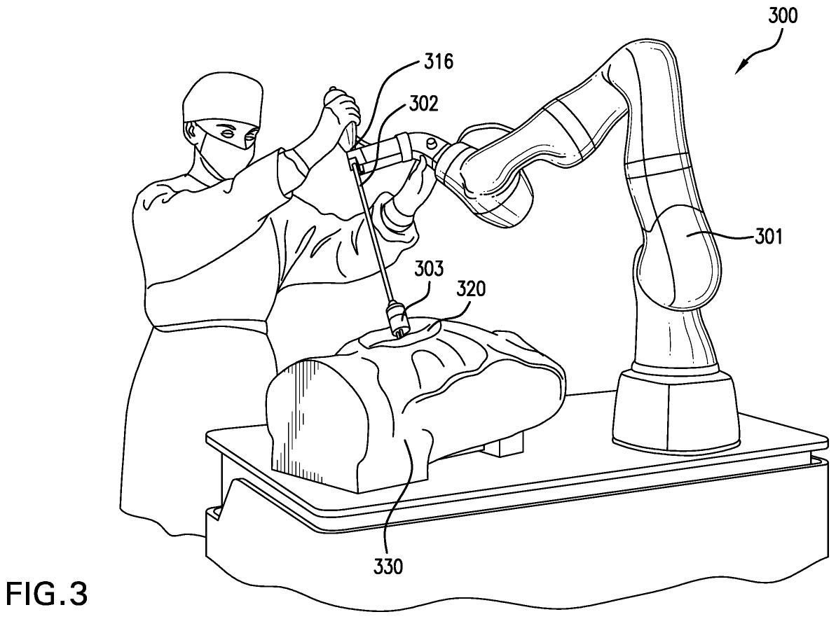

FIG. 3 illustrates a robotic arm system 300 for performing laparoscopic surgery according to an embodiment of the present disclosure. The robotic arm system 300 includes a robotic arm 301 affixed to a base at a proximal end. The robotic arm 301 further includes a surgical instrument 302 at the distal end of the robotic arm. The surgical instrument 302 includes a proximal end 316 and a distal end. In various embodiments, the proximal end 316 is affixed to the distal end of the robotic arm. In various embodiments, the proximal end 316 may be any point along a shaft of the surgical instrument 302. In FIG. 3, the surgical instrument 302 is inserted through a trocar 303 that is positioned within an incision 320 (e.g., a keyhole incision) in the patient 330. The surgical instrument 302 includes a wristed laparoscopic tool (not shown) at the distal end of the surgical instrument 302. The wristed laparoscopic tool may be any of the wristed laparoscopic tools described herein. In various embodiments, the three-dimensional position of the trocar 303, represented as P_{Trocar}=[x_{Trocar} y_{Trocar} z_{Trocar}]^{T}, is a known value.

In various embodiments, the pose, position information, and/or joint angles of the wristed laparoscopic tool may be used to plan surgical maneuvers in fully or partially automated surgical procedures (e.g., trajectory planning algorithm). In various embodiments, the pose, position information, and/or joint angles may be used to present a virtual indication of the wristed laparoscopic tool on a display to a user (e.g., a surgeon). For example, a virtual 3D laparoscopic tool may be displayed along with a 3D virtual anatomy (e.g., 3D anatomical atlas or 3D reconstruction of patient imaging) to allow a surgeon to visualize a procedure without seeing the physical laparoscopic tool. In various embodiments, the pose, position information, and/or joint angles may be used in collision detection, e.g., of a surgical instrument for a first robotic arm with another surgical instrument of a second robotic arm or with anatomical structures in the workspace.

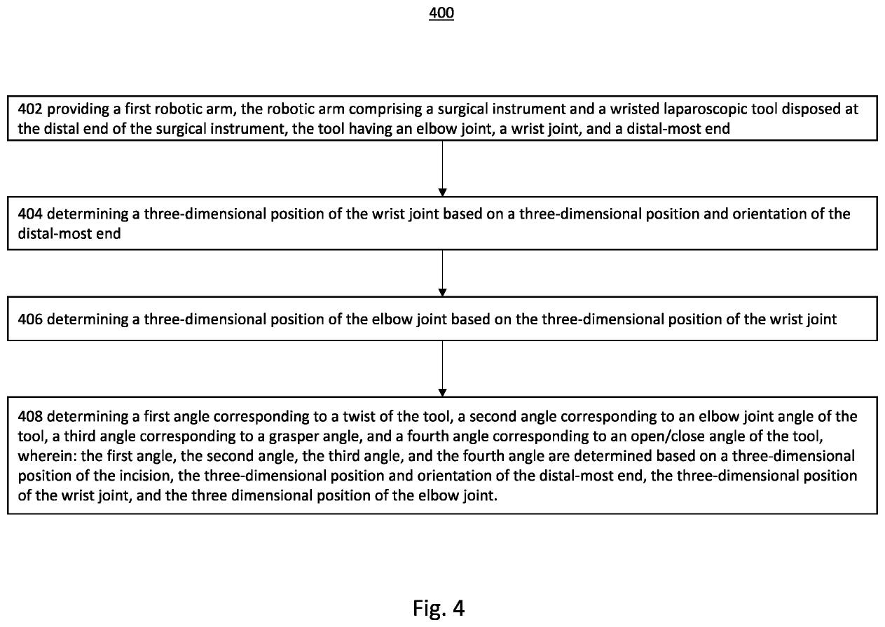

FIG. 4 illustrates a flowchart of a method 400 for determining joint angles of a wristed laparoscopic tool according to an embodiment of the present disclosure. At 402, a first robotic arm is provided. The robotic arm includes a surgical instrument and the tool disposed at a distal end of the instrument. The surgical instrument is configured to be inserted into an incision in a patient. A trocar may be inserted into the incision. The wristed laparoscopic tool has an elbow joint, a wrist joint, and a distal-most end. At 404, a three-dimensional position of the wrist joint is determined based on a three-dimensional position of the distal-most end. At 406, a three-dimensional position of the elbow joint is determined based on the three-dimensional position of the wrist joint. At 408, a first angle corresponding to a twist of the tool, a second angle corresponding to an elbow angle of the tool, a third angle corresponding to a wrist angle, and a fourth angle corresponding to an open/close angle of the tool are determined. The first angle, the second angle, the third angle, and the fourth angle are determined based on a three-dimensional position of the trocar, the three-dimensional position of the distal-most end, the three-dimensional position of the wrist joint, and the three dimensional position of the elbow joint.

In various embodiments, the algorithm inputs for determining end effector tool orientation error may include, for example, trocar position, abdominal cavity size and position, and desired end effector tip orientation. In various embodiments, error in the end effector orientation may be determined for two or more potential incision sites and the errors may be compared to determine an optimal incision site for a particular surgical procedure. It will be appreciated that in certain devices as described herein, an error metric grows with one or more of the first, second, and third angles. It will also be appreciated that the methods described herein are suitable for use with alternative error metrics and alternative relationships between individual angles and overall error.

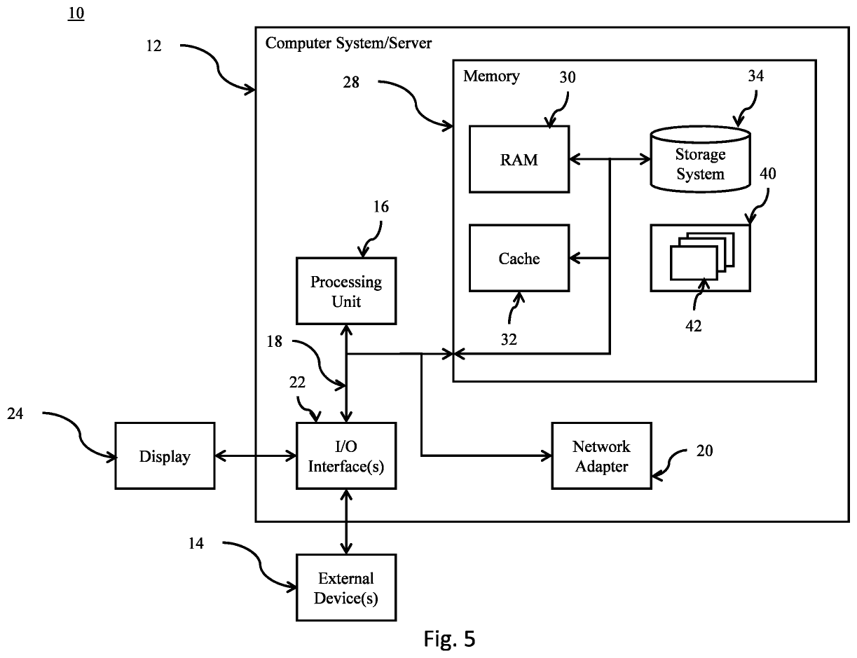

Referring now to FIG. 5, a schematic of an exemplary computing node is shown that may be used with the computer vision systems described herein. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 5, computer system/server 12 in computing node 10 is shown in the form of a general-purpose computing device. The components of computer system/server 12 may include, but are not limited to, one or more processors or processing units 16, a system memory 28, and a bus 18 coupling various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

In other embodiments, the computer system/server may be connected to one or more cameras (e.g., digital cameras, light-field cameras) or other imaging/sensing devices (e.g., infrared cameras or sensors).

The present disclosure includes a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In various embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In various alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1.-45. (canceled)

46. A method, comprising:
(a) inserting a robotic arm into a trocar positioned at an incision site of a patient, wherein the robotic arm comprises a surgical instrument and a tool disposed thereon, wherein the tool comprises an elbow joint and a wrist joint;
(b) determining (i) a three-dimensional (3D) position of the wrist joint based on a three-dimensional position and orientation of the tool and (ii) a three-dimensional position of the elbow joint based on the three-dimensional position of the wrist joint;
(c) determining (iii) a first angle corresponding to a twist of the tool, (iv) a second angle corresponding to an elbow angle of the tool, (v) a third angle corresponding to a wrist angle of the tool, and (vi) a fourth angle corresponding to an open/close angle of the tool, based at least in part on the three-dimensional position of the wrist joint and the three dimensional position of the elbow joint; and
(d) generating a virtual representation of a surgical scene that is accessible via the incision site, wherein the virtual representation comprises a virtual model of the surgical instrument and a virtual anatomy of one or more anatomical structures in the surgical scene, wherein the virtual representation provides a real-time visualization of a procedure involving the surgical instrument and the one or more anatomical structures.

47. The method of claim 46, wherein the real-time visualization provides a field of view for visualizing the procedure without seeing a physical tool or instrument.

48. The method of claim 46, wherein the virtual anatomy comprises a 3D anatomical atlas or a 3D reconstruction based on one or more images of the one or more anatomical structures.

49. The method of claim 46, further comprising, prior to (a), positioning the trocar at the incision site of the patient, wherein the position of the trocar is (i) specified by a surgeon or (ii) determined using a trajectory planning algorithm.

50. The method of claim 46, wherein at least one of the first angle, the second angle, the third angle, and the fourth angle is determined based on a three-dimensional position of the trocar and a three-dimensional position and orientation of a distal end of the tool.

51. The method of claim 46, further comprising, subsequent to (d), planning and executing one or more surgical maneuvers for the procedure using the virtual representation, wherein the procedure comprises a fully or partially automated surgical procedure.

52. The method of claim 46, wherein (c) further comprises determining a pose of the tool based at least in part on a three-dimensional position of the trocar and at least one of the first angle, the second angle, the third angle, or the fourth angle.

53. The method of claim 52, further comprising planning one or more surgical maneuvers based on the pose of the tool.

54. The method of claim 46, wherein the three-dimensional position of the wrist joint is determined based on a three-dimensional position of the trocar and a pose of a distal end of the tool.

55. The method of claim 46, wherein the three-dimensional position of the wrist joint is determined using a three-dimensional position of the distal end of the tool, a linear distance between the distal end of the tool and the wrist joint, and a directional unit vector pointing from the distal end of the tool to the wrist joint.

56. The method of claim 46, wherein the three-dimensional position of the elbow joint is determined based on an intersection of a circle centered at the three-dimensional position of the wrist joint and a plane defined by a first vector representing a rotation axis of the wrist joint and a second vector representing a line connecting the wrist joint to the incision site, wherein the circle comprises a radius equal to a linear distance between the three-dimensional position of the wrist joint and the three-dimensional position of the elbow joint.

57. The method of claim 56, wherein the intersection of the circle and the plane defines two or more solutions for the three-dimensional position of the elbow joint, wherein the method further comprises selecting at least one solution of the two or more solutions that does not violate one or more joint limits of the tool.

58. The method of claim 46, further comprising generating a planned surgical path for the surgical instrument using the three-dimensional position of the wrist joint, the three-dimensional position of the elbow joint, the first angle, the second angle, the third angle, and the fourth angle.

59. The method of claim 46, further comprising determining whether the surgical instrument will collide with an anatomical structure or another robotic arm, using the three-dimensional position of the wrist joint, the three-dimensional position of the elbow joint, the first angle, the second angle, the third angle, and the fourth angle.

60. The method of claim 46, wherein the first angle is determined based on a transformation matrix of the elbow joint with respect to a distal end of the tool.

61. The method of claim 46, wherein the second angle is determined based on the three-dimensional position of the wrist joint, the three-dimensional position of the elbow joint, and a three-dimensional position of the trocar.

62. The method of claim 46, wherein the third angle is determined based on (i) an angle between a line defined from the three-dimensional position of the elbow to the three-dimensional position of the wrist and a first gripping member of the tool, and (ii) an angle between a line defined from the three-dimensional position of the elbow to the three-dimensional position of the wrist and a second gripping member of the tool.

63. The method of claim 46, wherein the fourth angle is determined based on (i) an angle between a line defined from the three-dimensional position of the elbow to the three-dimensional position of the wrist and a first gripping member of the tool, and (ii) an angle between a line defined from the three-dimensional position of the elbow to the three-dimensional position of the wrist and a second gripping member of the tool.

64. The method of claim 46, further comprising providing (i) positional information for the elbow or wrist joints, (ii) the first, second, third, and/or fourth angles, and (iii) a pose of the tool to a control system and using the control system to move at least one of the elbow or wrist joints to a target angle to actuate the surgical instrument.

65. The method of claim 64, further comprising using the control system to move the wrist joint, the elbow joint, or the tool to a target position or pose to perform one or more steps of the procedure.
